(19) United States
(12) Patent Application Publication
(10) Pub. No.: US 2007/0088271 A1
(43) Pub. Date: Apr. 19, 2007

(54) Medication device

(76) Inventor: Cynthia C. Richards, Philadelphia, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 11/546,981

(22) Filed: Oct. 10, 2006

Related U.S. Application Data
(60) Provisional application No. 60/727,638, filed on Oct. 18, 2005.

(51) Int. Cl. A61M 1/00

(52) U.S. Cl. 604/151; 604/66; 604/890.1

(57) ABSTRACT

A dispenser for medicaments comprises a first metering pump for insulin and a second metering pump for glucose or glucagon. A controller for both pumps is programmed to maintain a basal supply of insulin, and is responsive to a signal from a separate glucometer to dispense additional insulin or glucose or glucagon as appropriate.

### FIELD OF THE INVENTION

The invention relates generally to a device for medication of a patient, and especially, but not exclusively, to a device for the subcutaneous administration of medicaments such as insulin and glucagon to diabetics.

### BACKGROUND OF THE INVENTION

It is known in the treatment of insulin-dependent diabetes to provide the patient with a metering pump that dispenses an insulin solution through a cannula in the patient's skin. One commercially available insulin pump, sold by Medtronic, Inc. under the name Minimed Paradigm, has a combined pump and electronic controller unit, that may be worn by the patient about his or her person. The pump unit is connected by a flexible tube to a cannula mounted on a holder attached by adhesive to the patient's skin, usually on the abdomen. The cannula passes through the skin. The pump unit comprises a cylinder with a piston driven by an electric motor that acts as a combined reservoir and metering pump for the insulin. The pump unit dispenses insulin at a basal rate, typically of the order of 0.1 units per second.

In addition, a separate blood glucose meter, also known as a “glucometer,” may be used to measure the blood glucose level at intervals. If the blood glucose level is too high, the glucometer sends a signal to the controller of the pump unit, which immediately dispenses an additional dose or “bolus” of insulin calculated to cause the body to metabolize the excess glucose. The glucometer communicates with the pump unit by a wireless link, such as a radio or infrared link.

These devices are very convenient for the patient. Because the glucometer is separate, the controller and pump unit can be made very compact, so that it is not obtrusive to wear. Because the single cannula for the insulin supply is the only permanent penetration of the patient's skin, and the single tube to the cannula is the only connection to the pump unit, the inconvenience to the user is kept to a minimum. The separate glucometer can be put away in the patient's pocket or purse when it is not in use.

However, if the blood glucose level is too low, existing insulin pumps cannot correct that. Merely reducing the rate of the basal insulin flow would not produce a quick enough effect. It is therefore necessary for the glucometer to alert the patient, and for the patient to take remedial action, typically by oral consumption of glucose or injection of glucagon. Unfortunately, low blood glucose levels tend to be accompanied by a loss of mental acuity, so that by the time a blood glucose test alerts the patient to the need for extra glucose the patient may not be thinking sufficiently clearly to administer the extra glucose correctly.

“Artificial pancreas” devices, in which a permanently implanted glucose sensor continually feeds glucose level information to a controller that operates both glucagon and insulin metering pumps, are proposed in U.S. Pat. No. 4,515,584 (Abe et al.) and U.S. Pat. No. 5,474,552 (Palti). However, the implanted glucose sensor is undesirable in a device that is to be used for non-hospitalized patients in everyday life.

There is therefore a need for a combined insulin and glucose pump that can be worn by a diabetic patient in everyday life, and that is not substantially more inconvenient to use than the existing insulin pumps currently in general use.

### SUMMARY OF THE INVENTION

According to one aspect of the invention, there is provided a combined insulin and glucose pump that comprises a first metering pump for insulin, a second metering pump for glucose or glucagon, and a controller for both pumps, wherein the controller is programmed to maintain a basal supply of insulin, and is responsive to a signal from a separate glucometer to dispense additional insulin or glucose/glucagon as appropriate.

In an embodiment, the separate glucometer measures the level of glucose in the patient's blood, and sends the measured value to the controller. The controller then determines whether the glucose level is too high, too low, or within an acceptable range. Where the glucose level is too high or too low, the controller activates the appropriate pump to dispense additional insulin or glucose/glucagon as appropriate. The controller may also determine how far outside the acceptable range, or how far from an optimum value, the glucose level is, and control the amount of additional insulin or glucose/glucagon accordingly.

According to another aspect of the invention, there is provided a refill for a medicament dispenser, comprising a first cartridge of insulin, a second cartridge of glucose or glucagon, a connector for connection to a subcutaneous infusion cannula, and flexible tubes connecting both cartridges to the connector.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1 shows somewhat schematically a first form of medicament dispenser according to the invention.

FIG. 2 shows a schematic exploded cross-section through an infuser head forming part of the dispenser shown in FIG. 1.

FIG. 3 is a view similar to FIG. 1 of a second form of medicament dispenser according to the invention.

FIG. 4 is a view similar to FIG. 1 of part of a third form of medicament dispenser according to the invention.

FIG. 5 is a view similar to FIG. 4 of part of a fourth form of medicament dispenser according to the invention.

FIG. 6 is a side view of a medicament cartridge forming part of a fifth form of medicament dispenser according to the invention.

FIG. 7 is a schematic view of part of a sixth form of medicament dispenser according to the invention.

### DETAILED DESCRIPTION OF THE EMBODIMENTS

Reference will now be made in detail to various embodiments of the present invention, examples of which are illustrated in the accompanying drawings.

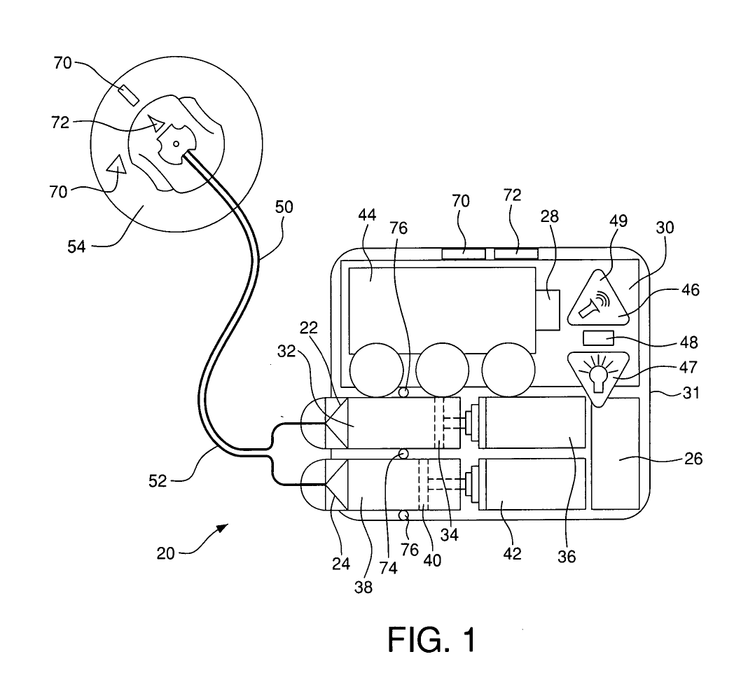

Referring initially to FIG. 1, a first form of medicament dispenser, indicated generally by the reference numeral 20, comprises an insulin pump 22 and a glucagon pump 24 powered by a battery 26 under control of a microprocessor 28 on a circuit board 30, all located within a housing 31.

The insulin pump 22 comprises a reservoir 32 for insulin in the form of a cylinder with a piston 34 that can be advanced in small, precisely controlled increments by a motor 36. The glucagon pump 24 comprises a reservoir 38 for glucagon in the form of a cylinder with a piston 40 that can be advanced in small, precisely controlled increments by a motor 42. For example, the motors 36 and 42 may be stepping motors that advance the piston by a screw or reduction drive.

The reservoirs 32, 38 may be disposable cartridges that are supplied to the patient already filled with appropriate solutions of insulin and glucagon. Alternatively, the reservoirs 32, 38 may be refillable by the patient, or a person attending the patient, from separate containers of insulin and glucagon solutions. Preferably, the reservoirs 32, 38 are of different shapes or sizes, and correspondingly shaped or sized seats are provided within the housing 31, so that neither reservoir can be inserted in the seat intended for the other reservoir and the pump operated. For example, one of the reservoirs 32, 38 may be formed with an exterior of circular cross-sectional shape, and the other may be formed with an exterior of square cross-sectional shape. If the diameter of the circular shape is greater than the length of side of the square shape, but the diameter of the circular shape is less than the diagonal of the square shape, then each shape will fit into a correspondingly shaped seat that the other cannot fit into. For example, if both reservoirs 32, 38 are of circular cylindrical shape, one may be longer and the other may be wider. Differentiating the shapes in this way may slightly increase the cost of manufacture of both the dispenser 20 and the reservoirs 32, 38 but considerably reduces the risk of the wrong reservoir being loaded into one or both of the pumps 22, 24.

Where the reservoirs 32, 38 are refillable, they may be configured as syringes, with a manually operable plunger fixed to the piston 34, 40 by an actuating rod 43 and projecting from the reservoir. The motors 36, 42 may then be arranged to drive the plungers. Where the reservoirs 32, 38 are disposable pre-filled cartridges, the pistons 34, 40 may be arranged not to project from the body of the reservoir when the reservoir is full. The actuating rods 43 may then be permanently attached to the motors 36, 42.

The circuit board 30 is provided with a display screen 44 and controls 46, which may be push buttons, on the outside of the housing 31. The circuit board 30 is also provided with a receiver 48 for wireless signals from a separate glucometer (not shown). The display screen and controls may be used for programming the microprocessor 28 with information to enable the microprocessor 28 to estimate an appropriate dosage of insulin and/or glucagon in response to blood glucose level readings received by the wireless receiver 48. The information may include general information, such as the patient's basal metabolism and responsiveness to insulin and glucagon, and specific information, such as amounts and types of food that the patient is about to eat or has just eaten. Algorithms for calculating insulin and glucagon dosages in artificial pancreas devices are known, see for example U.S. Pat. No. 6,572,545 to Knobbe, which is incorporated herein by reference, and may be adapted to the characteristics of the present dispenser 20. In particular, the fact that calculations are carried out only at discrete intervals, when the blood glucose level is received from the glucometer, simplifies the computations, although continuous monitoring allows more precise control. The display 44 may also display information on the status of the dispenser 20 and of the patient. The controls 46 may also include a light switch 47 to illuminate the display screen 44 for use in the dark, and a panic button 49 to emit an audible or other emergency signal.

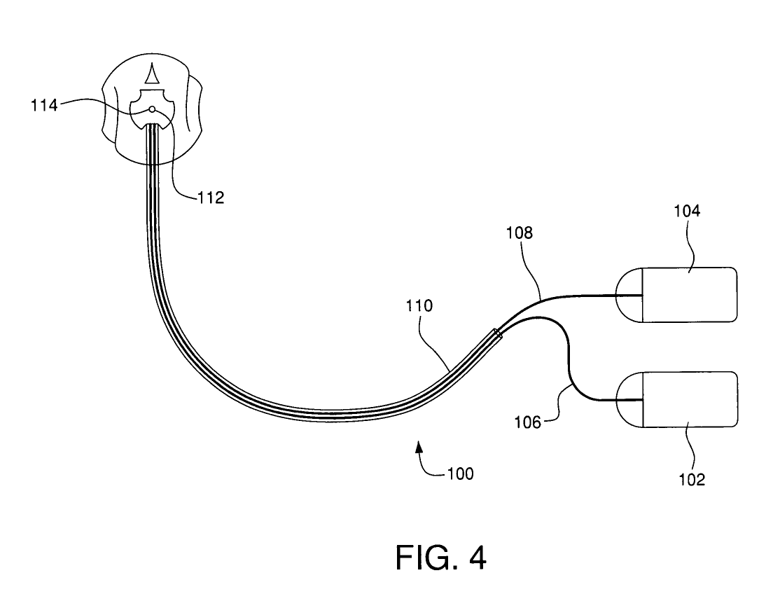

The output from each of the pumps 22, 24 is connected to a hollow tube 50, 52. The other ends of the tubes 50, 52 are connected to an infusion head 54. The tubes 50, 52 are formed as a dual-lumen tube of figure-8 cross-section. Alternatively, the tubes 50, 52 may be separate tubes within an outer sheath 110 (see FIG. 4). The ends of the tubes 50, 52 nearest the housing 31 are separated so that each tube can be connected to the correct pump 22, 24. The tubes 50, 52 are joined for the major part of their length, so that there is effectively only a single flexible line 56 connecting the housing 31 to the infusion head 54, which flexible line 56 may be only slightly less flexible than the insulin tube of a comparable conventional insulin pump. Thus, the inconvenience to the patient from the line 56, and the risk of the line 56 snagging on other objects, need not be substantially greater than with the insulin tube of a comparable conventional insulin pump.

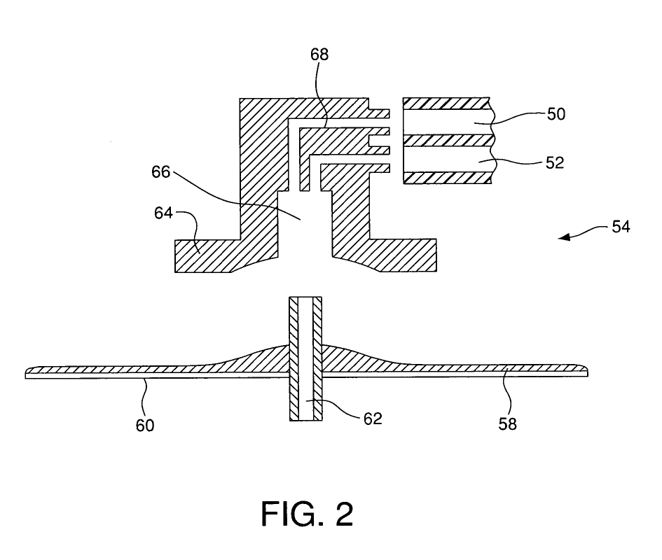

Referring now also to FIG. 2, the infusion head 54 comprises a base in the form of a disk 58 that is attached to the patient's skin with adhesive 60, and that has mounted in the center of the disk 58 a cannula 62 to penetrate the patient's skin. The cannula 62 may be placed by threading a sharp metal probe through the cannula 62, inserting the probe with the cannula into the skin, and then removing the probe, leaving the cannula in place. The cannula 62 may be of hypoallergenic plastic material. The infusion head 54 further comprises a connector 64 that has a socket 66 to receive the exposed end of the cannula 62, and a pair of ports 68 for the tubes 50, 52 in communication with the socket 66. In use, the connector 64 may be releasably secured to the disk 58 by a fastening (not shown in detail) such as a bayonet connection. The disk 58 and the connector 64 may then be provided with visible symbols that are aligned to select a “locked” and an “unlocked” alignment of the disk 58 and the connector 64. The bayonet connection permits the connector 64 and the base disk 58 to be separated and brought together in the unlocked alignment, and holds the connector and the base disk securely together in the locked alignment.

In an embodiment, the tubes 50, 52 are substantially identical, so it is not important which tube is connected to which pump. Alternatively, the tubes 50, 52 may be provided with connectors, or a common connector, that can only be connected to the pumps 22, 24 one way round. If the tubes 50, 52 may be detached from the pumps 22, 24 and reattached with medicament remaining in the tubes, then it may be more important to ensure that each tube 50, 52 is reattached to the same pump 22, 24 that tube 50, 52 was detached from.

In use, the cannula 62 is inserted into the patient's skin and is secured to the skin by the adhesive 60, if the cannula is not already in place. As a compromise between the discomfort of inserting the cannula 62 and the risk of forming scar tissue or infection if the cannula is left in place too long, it is presently preferred to remove the cannula, and insert a new cannula at a different site, every 2 or 3 days. By releasing the connector 64 from the base disk 58, the medicament dispenser 20 can be temporarily removed, for example, while the patient takes a shower, without removing the cannula 62 and the base disk 58. When the cannula 62 is inserted, conventional procedures, such as the use of an alcohol swab or the like to disinfect the skin, may be applied.

The reservoirs 32, 38 are filled with insulin and glucagon, or other appropriate medicaments, and are loaded into the appropriate pumps 22, 24. The tubes 50, 52 are connected to the pumps 22, 24. Depending on the attachment used, the tubes 50, 52 may be connected to the reservoirs 32, 38 before the reservoirs are loaded into the pumps 22, 24. The tubes 50, 52 may then terminate in connectors (not shown in detail) that serve to mount both the tubes and the reservoirs in the pumps. The pumps 22, 24 are then primed by pumping from both pumps a quantity of medicament solution equal to the known internal volume of the tubes 50, 52. For this purpose, the processor 28 may be pre-programmed, or programmed by the user, with the size of the tubes 50, 52. In an embodiment, the diameter of the tubes 50, 52 is fixed, and is pre-programmed, and the length of the tubes 50, 52 is chosen and input by the patient. The connector 64, with the tubes 50, 52 attached, is then secured to the disk 58, forming a connection between the ports 68 and the cannula 62. The dispenser 20 is then further primed to fill the cannula 62 with medicament solution. Where, for example, the dispenser 20 is to be used to supply both basal and bolus insulin, and bolus glucagon, the cannula 62 may be primed with insulin, and basal insulin supply may then start immediately.

Bolus dispensing of either insulin or glucagon may be caused by the patient inputting at the controls 46 a command to dispense a bolus or information about food consumption from which the microcomputer 28 is programmed to determine that a bolus is appropriate. Bolus dispensing of either insulin or glucagon may also be caused by the receiver 48 receiving a blood glucose level from the separate glucometer, and the microcomputer 28 calculating that a rapid increase in either glucose or insulin is desirable. Smaller departures from the desired blood glucose level may be corrected by adjusting the basal insulin rate.

As shown in FIG. 2, there are no valves between the separate ports 68 that connect to the tubes 50, 52 and the single cannula 62. It is believed that valves are unnecessary, because the small diameter of the ports 68, combined with the use of piston pumps 22, 24, which are positive-displacement pumps, will sufficiently prevent the medicament from either tube 50, 52 flowing upstream into the port 68 connected to the other tube 52, 50. However, non-return valves may be provided if desired.

The microcomputer 28 may monitor the function of the dispenser 20, and may generate an alert on the display 44, a visible signal on a lamp 70, and/or an audible signal on a beeper or siren 72, when a malfunction occurs. The lamp 70 may also be controlled by the light button 47, and the siren or beeper may also be actuated by the panic button 49. Malfunctions to be monitored may include a kink in the tubes 50, 52 preventing the insulin or glucose/glucagon from flowing freely, which may be detected by pressure upstream of the kink or resistance to movement of the pistons 34, 40. An empty reservoir 32, 38 may be detected by the piston 34, 40 ceasing to move as the piston reaches the end of the reservoir. A reservoir 32, 38 that is low but not yet empty may be detected by counting or measuring the advance of the piston 34, 40 along the reservoir. A low battery may be detected by monitoring the battery voltage or other appropriate factors. Systems for monitoring the battery level are generally available for most generally available forms of battery. Other malfunctions may be monitored and detected in various ways, including ways that are already known in this or other arts.

When a new supply of glucose/glucagon or insulin is loaded by replacing or refilling the cartridge or other reservoir 32, 38, the dispenser may test to confirm that the correct medicament has been loaded in the correct reservoir. For example, glucose may be distinguished from insulin spectroscopically, by shining through the reservoir 32, 38 from lamps 74 to detectors 76 one or more beams of monochromatic light at frequencies at which one of glucose and insulin has an absorption peak and the other does not. The test can be triggered automatically by the act of replacing or refilling the reservoir 32, 38, and needs to be run only once for a short period. The load on the battery 26 to power the lamps 74 and detectors 76 therefore need not be large.

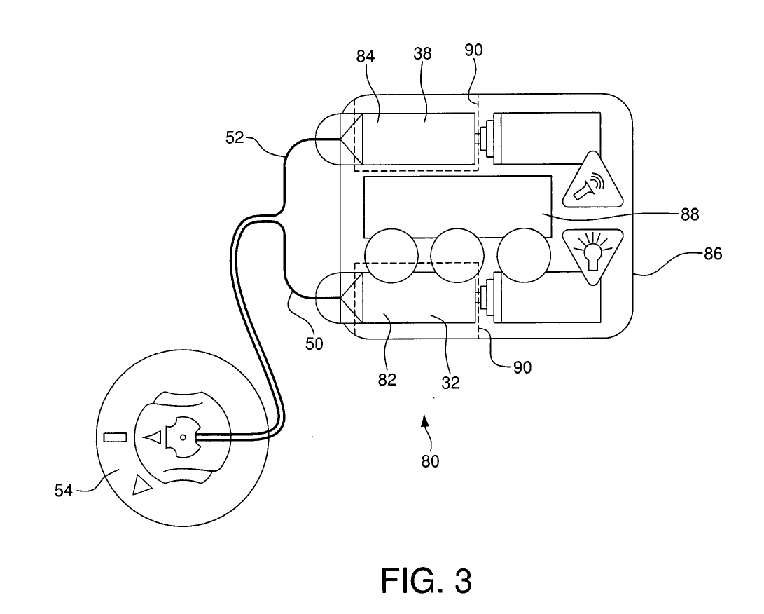

In the dispenser 20 shown in FIG. 1, the two pumps 22, 24 are close together on one side of the housing 31. Referring now to FIG. 3, an alternative embodiment of medicament dispenser 80 is substantially the same as the first form of dispenser 20, except that the two pumps 82, 84 are on opposite sides of the housing 86, with the circuit board 88 carrying the control and display electronics between the two pumps. The arrangement shown in FIG. 3 may make it easier to insert and remove the medicament reservoirs 32, 38, because each reservoir is at a side of the housing 86 and can be exposed by removing a cover 90 that forms parts of two or three faces of the generally rectangular housing 86. In comparison, in FIG. 1 one of the reservoirs 32 is in the interior of the housing 31 and is less easily accessible. On the other hand, the points of attachment of the tubes 50, 52 to the pumps 82, 84 in the dispenser 80 shown in FIG. 3 are further apart than the points of attachment of the tubes to the pumps 22, 24 in the dispenser 20 shown in FIG. 1. Thus, in FIG. 3 there may be either a larger and more complicated connector at the end of the tubes 50, 52 or a loop formed by the separated parts of the tubes 50, 52 that could snag on other objects.

Referring now to FIG. 4, in a third form of dispenser 100 the medicament reservoirs 102, 104 are disposable cartridges attached by the manufacturer to flexible tubes 106, 108, which as shown in FIG. 4 are separate tubes in a sleeve 110. The other ends of the flexible tubes 106, 108 are attached by the manufacturer to a connector 112 that is substantially the same as the connector 86 shown in FIG. 2. The reservoirs 102, 104 are already in communication with the tubes 106, 108. The tubes 106, 108 are sealed off by a removable seal 114 where the tubes open out through the connector 112. The patient thus obtains a pre-packaged unit, with everything from reservoirs 102, 104 to connector 112 in a single assembly in a single package. The patient simply inserts the cartridges 102, 104 into the pumps 22, 24 or 82, 84 of a dispenser that may otherwise be the same as the dispenser 20, 80 shown in FIG. 1 or FIG. 3, and in the interests of conciseness is not further described or shown in FIG. 4. The patient then merely removes the seal 114 from the connector 112, attaches the connector 112 to a disk 58 that may be substantially the same as the one shown in FIG. 2, and the dispenser 100 is ready to use. The dispenser 100 is especially suitable for use by inexperienced patients because no filling or assembly of the reservoirs 102, 104 is required. Apart from removing the seal 114 and clipping the connector 112 onto the disk 58, no manipulation of the sterile parts of the dispenser 100 by the patient is required, and there is little risk of contamination of the medicament.

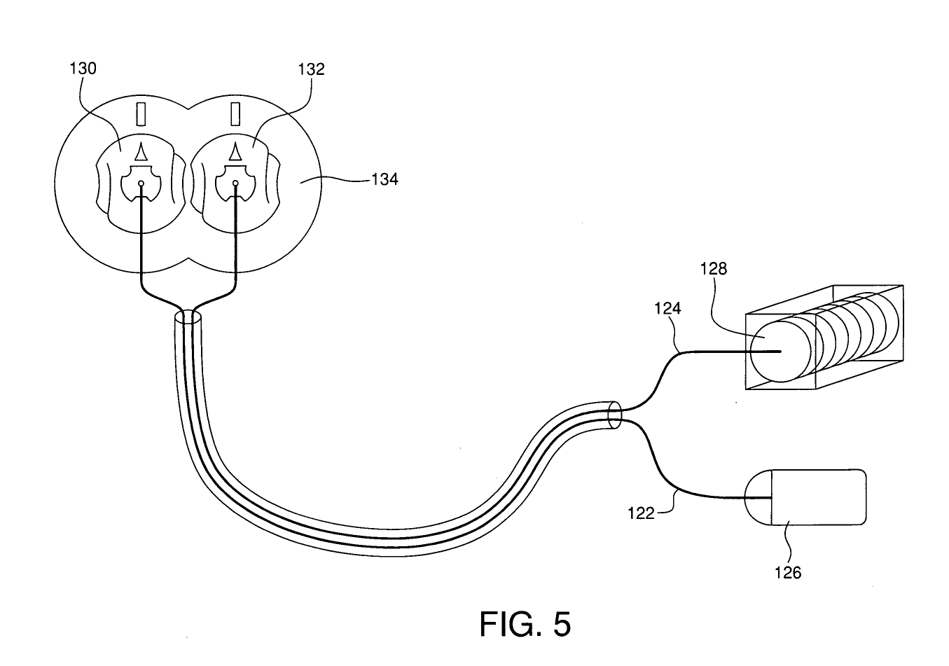

Referring now to FIG. 5, in a fourth form of dispenser 120, the tubes 122, 124 from the insulin reservoir 126 and the glucagon reservoir 128 are attached to separate connectors 130, 132 instead of to a single connector 64. As shown in FIG. 5, the connectors 130, 132 are attached to a single base 134 that has two spaced-apart cannulae 62 mounted in it. The base 134 is in the form of two overlapping disks each centered on a respective one of the cannulae 62, but could alternatively be of an oval or oblong shape. The cannulae 62 are positioned as close together as is practical without the connectors 130, 132 obstructing each other. In FIG. 5, connectors of a commercially-available design are used, which are circular with concave recesses in two opposite sides for easy gripping by the patient. The connectors 130, 132 are positioned so that either connector can be turned from the locked to the unlocked position by allowing the circular part of one connector to enter the recess of the other connector. Alternatively, the connectors 130, 132 may be positioned far enough apart that they can rotate independently without interfering, or connectors of a smaller design may be used.

In the dispensers 20, 80, 100 shown in FIGS. 1 to 4, if a change is made from dispensing insulin to dispensing glucagon, or vice versa, a volume of the first medicament equal to the volume of the cannula 62 is expelled after pumping of the second medicament starts, before the second medicament reaches the patient. It is presently believed this is not usually likely to be problematic, and can usually be compensated for by appropriately increasing the bolus volume of the second medicament that is dispensed. It is estimated that the volume of the cannula 62 typically corresponds to a few seconds' flow at the basal insulin rate of a typical patient. Alternatively, when a single bolus of glucose or glucagon interrupts the basal flow of insulin, the displacement of each medicament from the cannula when pumping of the other medicament starts may be regarded as canceling out. The dispenser 120 shown in FIG. 5 avoids this question, by using separate cannulae 62. On the other hand, the dispenser 120 shown in FIG. 5 imposes on the patient the discomfort of inserting two cannulae, and the inconvenience of a larger adhesive base 134 with two connectors 130, 132 projecting.

The base 134 shown in FIG. 5 may be positioned using a modified insertion device with two probes that can insert both cannulae 62 in a single operation, either simultaneously or in succession. Such an insertion device can reduce the difficulty for the patient of ensuring that the two cannulae are the correct distance apart for the base 134 to adhere smoothly to the patient's body. Some patients may consider that inserting two cannulae in a single operation is less unpleasant than inserting two cannulae in separate operations. On the other hand, the need to ensure clean insertion of both cannulae at once may restrict positioning of the base 134 on curved parts of the body. Some patients may prefer separate insertions, as providing greater psychological assurance that both cannulae have been properly inserted. A mechanism that inserts the two cannulae in distinct actions, for example, half a second apart, but in response to a single actuation by the patient, may be preferred by some patients.

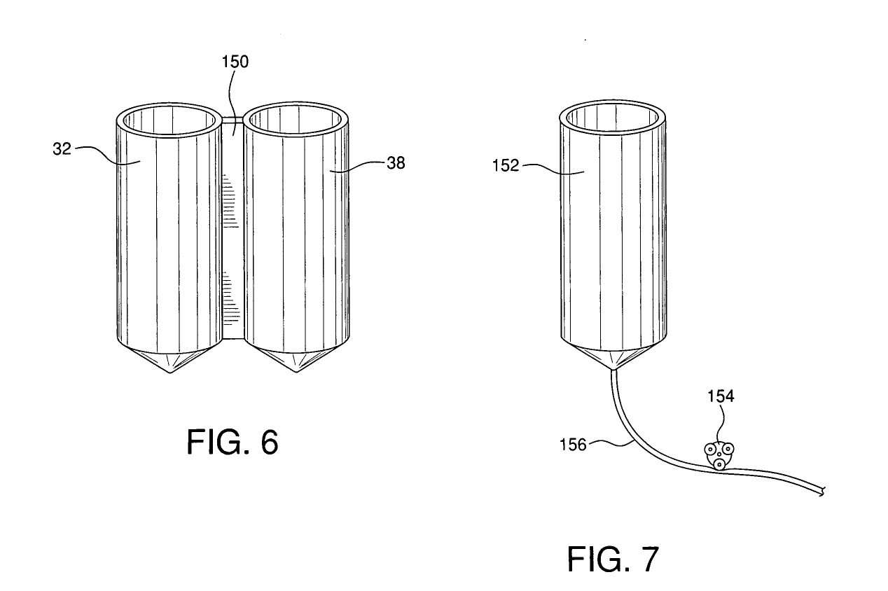

Referring now to FIG. 6, a fifth form of dispenser according to the invention is similar to the dispenser 20 shown in FIG. 1, except that the insulin and glucagon reservoirs 32, 38 are in a single unit 150. As shown in FIG. 6, the two reservoirs 32, 38 may be spaced apart so as to correspond as nearly as possible to the positions of the reservoirs 32, 38 in FIG. 1, enabling the single unit 150 to be used with the dispenser 20 with little or no modification to the dispenser. The unit 150 is preferably asymmetrical, to prevent its being inadvertently installed the wrong way round. The single unit 150 may be more convenient than the separate reservoirs 32, 38 shown in FIG. 1, because only one unit has to be changed instead of two. However, if the ratio of the patient's insulin consumption to glucose consumption does not match the ratio of the contents of the two reservoirs 32, 38 the single unit 150 may be less economical, because both reservoirs must be changed as soon as one reservoir is exhausted, and any remaining contents of the other reservoir are wasted.

Referring now to FIG. 7, in a sixth form of dispenser, the reservoirs 152 for glucagon and insulin are separate from the pumps 154. The pump may be of a suitable type, including conventional types. For example, the pump may be a reciprocating displacement pump that draws fluid from the reservoir 152 and expels it to the tube 156 leading to the infusion head 54, 112, 130, 132. However, if the pump is disposable the cost may be significant, and if the pump is reusable then cleaning and sterilization may be inconvenient. A peristaltic pump 154 that can act directly on the tube 156 is therefore presently preferred. With a peristaltic pump, the moving parts do not come in contact with the fluid being pumped, and cleaning issues can therefore be greatly reduced. A construction similar to that shown in FIG. 4, with a disposable tube 156 attached by the manufacturer to a disposable reservoir 152, is then possible.

Because the reservoir 152 shown in FIG. 7 does not form part of the pump mechanism, there is more freedom of design than in FIG. 1. For example, a flattened shape, although possible for the reservoir 32 or 38 in FIG. 1, is not presently preferred because the pressure within the reservoir during pumping may cause the flattened sides to deflect away from the piston 34, 40. To avoid leakage, extra attention may be paid to stiffening the reservoir or to piston seals that can accommodate the deflection. The reservoir 152 is under suction rather than pressure, and deflection of the flattened sides towards a piston or other movable closure is less likely to result in leakage. In addition, any movable closure is not used to deliver working force, but merely to take up the space vacated as the contents are withdrawn and ensure that a void does not occur at the outlet. As a result, a flexible reservoir 152, or a partially flexible reservoir 152 such as a reservoir closed by a rolling membrane, is possible.

The greater freedom of design of the reservoir 152 allows greater freedom of design of the pump unit as a whole, and may allow a more compact, or less obtrusively shaped, unit. For example, in the device 20 shown in FIG. 1, the volume of the reservoirs 32, 38 is constrained by the need to store a reasonable amount of insulin and glucagon. The reservoirs 32, 38 are of circular cross-section. The length of the pump 22, 24 including the motor 36, 42 is at least twice the length of the reservoir 32, 38, to provide space for the actuating rod 43 when the reservoir is full. As a result, lower limits are imposed on the length and the thickness of the housing 31, 86 even though the volume within the housing may not be full. With the reservoirs 152 of FIG. 7, a more efficient use of volume is possible, allowing a housing 31, 86 that is thinner and/or shorter than the housing 31 shown in FIG. 1. In particular, a housing thinner than is practical in FIG. 1 may be attractive to some users, because it can more easily be concealed under clothing.

In a further modification of the embodiment shown in FIG. 7, the reservoir 152 and the peristaltic pump 154 may be combined, by providing a flexible reservoir that is squeezed between a pair of rollers to expel the fluid from the reservoir in a controlled manner.

In a further modification of the embodiment shown in FIG. 7, the reservoirs 152 for insulin and glucagon may be combined in a single unit, similarly to the unit 150 shown in FIG. 6.

It will be apparent to those skilled in the art that various modifications and variations can be made in the present invention without departing from the spirit or scope of the invention. For example, the dispenser 120 shown in FIG. 5 may be used with two separate disk bases 58 similar to those shown in FIGS. 1 to 4 instead of one double base as shown in FIG. 5.

For example, the sleeve 110 may be omitted from the tubing 106, 108 or 122, 124 shown in FIGS. 4 and 5, allowing the two tubes 106, 108 or 122, 124 to be positioned separately. In the dispenser 120 shown in FIG. 5 with two separate disk bases 58, the disk bases 58 can then be positioned independently. Either the dispenser 100 shown in FIG. 4 or the dispenser 120 shown in FIG. 5 can then be provided with two separate pump housings, one containing the insulin pump 22 and the other containing the glucagon pump 24, instead of the single housing 31, 86 shown in FIGS. 1 and 3.

The patient may then be provided with separate pre-packaged units, consisting essentially of insulin reservoir 126, tubing 122, and connector 130, or glucagon reservoir 128, tubing 124, and connector 132. These pre-packaged units can be replaced separately as refills, if a particular patient uses insulin and glucagon at rates that are not proportionate to the sizes of the reservoirs 126, 128. In addition, a single pre-packaged unit, for example, insulin reservoir 126, tubing 122, and connector 130, may be used separately as a refill for an existing insulin dispenser.

Embodiments are shown in the drawings with a single connector connecting both tubes 50, 52 to a single cannula 62, or with separate connectors connecting the tubes 122, 124 to separate cannulae 62. Alternatively, a single connector may connect the two tubes to separate but closely-spaced cannulae 62.

The embodiments described are programmed to dispense basal insulin, bolus insulin, and bolus glucagon. Alternatively, in an appropriate case, the dispenser may be programmed to dispense basal glucagon as well as basal insulin. The two medicaments may then be dispensed simultaneously, with a mixture of insulin and glucagon flowing through the cannula 62, or alternately, with each medicament in turn being dispensed for a short period. Alternatively, fluid comprising both insulin and glucagon may be used to provide basal glucagon and basal insulin in a predetermined mixture, and separate reservoirs for bolus insulin and/or bolus glucagon may also be provided. Other medicaments may be used instead of insulin and glucagon. For example, a solution of glucose may be used instead of glucagon.

Although several distinct embodiments have been shown and described, features from different embodiments may be combined in a single device. For example, any of the embodiments described may be used with refillable reservoirs or preloaded cartridges attached to the flexible tubes only when installed in the pump, as described with reference to FIG. 1, or cartridges pre-packaged with tubes and connectors, as described with reference to FIG. 4. For example, either the figure-8 tubing 50, 52 or the separate tubes in a sleeve 110 may be used in any of the embodiments except when it is desired to separate the tubes 106, 108 or 122, 124.

The cartridges or reservoirs 32, 38 may be of any convenient size. Most patients require more insulin than glucose or glucagon. It may therefore be preferred to have the insulin reservoir 32 larger than the glucose or glucagon reservoir 38, so that both reservoirs last for approximately the same length of time between refills. This is most significant in the case of a device such as the devices shown in FIGS. 4 and 6, where the reservoirs 32, 38 or 102, 104 are both changed at the same time. The size of the reservoirs 32, 38, etc. may be chosen to give a three-day supply for a typical patient under normal conditions, because that is a common duration for existing devices, and patients are used to the routine. By avoiding the need to accustom the patient to a new routine, the risk of errors during the transition from one medication supply device to another is reduced.

Modifications and variations may include the incorporation of technology hereafter to be developed. For example, a lithium ion battery is presently preferred as the battery 26, because of its small size and long life compared with generally available alternatives, although the lithium ion battery is comparatively expensive. However, advances in battery technology are being made continually. It is therefore expected that smaller, lighter, longer-lasting and/or less expensive batteries will become available in the future, and that in appropriate cases such improved batteries may be used instead of a lithium ion battery for the battery 26.

Thus, it is intended that the present invention cover the modifications and variations of this invention provided they come within the scope of the appended claims and their equivalents.

1. A dispenser for medicaments, comprising:
a first metering pump for insulin;
a second metering pump for glucose or glucagon; and
a controller for both pumps, wherein the controller is programmed to maintain a basal supply of insulin, and is responsive to a signal from a separate glucometer to dispense additional insulin or additional glucose or glucagon as appropriate.

2. A dispenser according to claim 1, further comprising a wireless receiver for receiving the signal from the separate glucometer.

3. A dispenser according to claim 1, further comprising a pair of flexible tubes arranged to be connected at one end to a respective one of the first and second metering pumps and at the other end to a connector for a cannula arranged to be inserted through the skin of a patient.

4. A dispenser according to claim 3, wherein both of the pair of flexible tubes are connected to a single connector for a single cannula.

5. A dispenser according to claim 3, wherein the flexible tubes are joined together over a major portion of their length.

6. A dispenser according to claim 1, wherein said first and second metering pumps are arranged to receive reservoirs of medicament of different configurations.

7. A dispenser according to claim 6 in combination with a first reservoir for insulin and a second reservoir for glucose or glucagon, wherein said first metering pump receives said first reservoir but not said second reservoir, and said second metering pump receives said second reservoir but not said first reservoir.

8. A dispenser according to claim 1 in combination with a first reservoir containing insulin and a second reservoir containing glucose or glucagon, wherein said first metering pump is arranged to dispense the contents of the first reservoir, and the second metering pump is arranged to dispense the contents of the second reservoir.

9. A dispenser according to claim 8, wherein one of said first and second reservoirs contains both insulin and glucose or glucagon, wherein the controller is arranged to dispense basal insulin and basal glucose or glucagon by dispensing fluid from the said one reservoir.

10. A dispenser according to claim 1, further comprising a third metering pump for a mixture of insulin and glucose or glucagon;
wherein the controller is programmed to maintain a basal supply of insulin and glucose or glucagon by the third metering pump, and is responsive to a signal from a separate glucometer to dispense additional insulin by the first metering pump or additional glucose or glucagon by the second metering pump as appropriate.

11. A refill for a medicament dispenser, comprising:
a first cartridge of insulin;
a second cartridge of glucose or glucagon;
at least one connector for connection to a subcutaneous infusion cannula; and
flexible tubes connecting both cartridges to the at least one connector.

12. A refill according to claim 11, wherein the first and second cartridges are of different configurations such that each cartridge can be non-interchangeably inserted into a respective one of first and second metering pumps with correspondingly different configurations.

13. A refill according to claim 11, comprising a said connector to which both cartridges are connected by the flexible tubes.

14. A refill according to claim 11, comprising two said connectors, and wherein the cartridge of insulin is connected to one of the connectors by one of the flexible tubes and the cartridge of glucose or glucagon is connected to the other connector by another of the flexible tubes.

15. A refill according to claim 11, wherein the first and second cartridges are formed in a single unit.

16. A refill for a medicament dispenser, comprising:
a first cartridge containing a medicament;
a connector for connection to a subcutaneous infusion cannula;
a flexible tube connecting the cartridge to the connector; and
a removable seal closing the flexible tube at the connector.

17. A refill according to claim 16, further comprising a second cartridge containing a second medicament and a second flexible tube connecting the second cartridge to the connector, and wherein the removable seal closes both flexible tubes at the connector.

18. A refill according to claim 16, wherein the first and second cartridges are of different configurations such that each cartridge can be non-interchangeably inserted into respective first and second metering pumps with correspondingly different configurations.

19. A refill according to claim 16, wherein one of the first and second cartridges contains insulin and the other of the first and second cartridges contains glucose or glucagons.

20. A refill according to claim 16, further comprising a second cartridge containing a second medicament, a second connector for connection to a second subcutaneous infusion cannula, a second flexible tube connecting the cartridge to the connector, and a second removable seal closing the second flexible tube at the second connector.

21. A refill according to claim 20, wherein the first and second cartridges are of different configurations such that each cartridge can be non-interchangeably inserted into respective first and second metering pumps with correspondingly different configurations.

22. A refill according to claim 20, wherein one of the first and second cartridges contains insulin and the other of the first and second cartridges contains glucose or glucagons.
